(19) United States
(12) Patent Application Publication
(10) Pub. No.: US 2026/0317287 A1
(43) Pub. Date: Oct. 15, 2026

(54) METHODS FOR PRODUCING FUROIC ACID DERIVATIVES, FURAN DERIVATIVES, PHTHALOCYANINE DERIVATIVES, AND ISOINDOLINE DERIVATIVES

(71) Applicant: DIC Corporation, Tokyo (JP)

(72) Inventors: Siongwan Foo, Kamisu-shi (JP); Hiroshi Ebato, Tokyo (JP); Fangxi Xiu, Qingdao (CN); Changjun Deng, Qingdao (CN)

(73) Assignee: DIC Corporation, Tokyo (JP)

(21) Appl. No.: 19/482,919

(22) PCT Filed: Jun. 25, 2023
(86) PCT No.: PCT/CN2023/102111

§ 371 Date: Nov. 10, 2025

(51) Int. Cl. C07D 307/68; C07D 209/44; C07D 307/36; C07D 307/89; C07D 403/04; C07D 403/14; C07D 487/22; C07D 493/18

(52) U.S. Cl. CPC C07D 307/68; C07D 209/44; C07D 307/36; C07D 307/89; C07D 403/04; C07D 403/14; C07D 487/22; C07D 493/18

(57) ABSTRACT

A method for producing a furoic acid derivative may include an oxidation step of oxidizing a furfural derivative to obtain a furoic acid derivative in the reaction system of pH 3-12. A method for producing a furan derivative may include a decarboxylation step of decarboxylating a furoic acid derivative to obtain a furan derivative without using a catalyst, using an oxide of mono- to tri-valent transition metal as a catalyst, or using a high boiling point solvent. A method for producing a furan derivative may include the oxidation step of obtaining a furoic acid derivative and the decarboxylation step of obtaining a furan derivative. A method for producing a phthalocyanine derivative or an isoindoline derivative may include obtaining a phthalocyanine derivative or an isoindoline derivative from the furan derivative obtained by any one of the methods for producing a furan derivative.

### TECHNICAL FIELD

The present disclosure relates to methods for producing furoic acid derivatives, furan derivatives, phthalocyanine derivatives, and isoindoline derivatives, respectively.

### BACKGROUND

Furan is used as a raw material in a variety of industries, including pigments, dyes, pharmaceuticals, agrochemicals, fragrances, polymers, and resins, and has many applications, especially in organic chemistry, where it is used as a solvent, chemical intermediate, and polymerization initiator, as well as a starting material in the synthesis of other organic compounds such as phthalocyanines.

For example, The global furan resins market was valued at US $15.66 billion in 2021, and it is expected to reach US $23.68 billion by 2029 with a CAGR of 5.3%. (see “Furan Resins Market—Global Industry Analysis by Type, Application, Region and Forecast (2022-2029)” [online], [Search Jun. 6, 2023] internet <URL: https://www.maximizemarketresearch.com/market-report/global-furan-resins-market/116002/>).

As demand for furan increases, the depletion of fossil raw material resources, greenhouse gas emissions, and other issues have prompted a smooth transition from fossil-derived furans to biomass-derived furans, and there is an urgent need to develop new, more efficient production technologies.

The production methods for furan using furfural, furoic acid, 5-hydroxymethylfurfural, butane, butadiene, cis-1,4-dihydroxy-2-butene, tetrahydrofuran, aldotetrose, or other compounds as a starting material are known. Furfural is the most widely used starting materials due to their structural usefulness. For example, NPL 1 discloses a method for producing furan using furfural as a starting material. NPL 2 discloses a method for producing furan using furoic acid as a starting material. It is known that furoic acid can be obtained using furfural as a starting material (see, for example, PTL 1, PTL 2, and NPL 3).

### CITATION LIST

### Patent Literature

PTL 1: CN109485624A
PTL 2: CN111217775A

### Non-Patent Literature

NPL 1: Chemical Communications, 2012, 48, 4253-4255
NPL 2: Chemistry European Journal, 2013, 19, 14034-14038
NPL 3: Angewandte Chemie, International Edition, 2016, 55, 36, 10806-10810

### SUMMARY

### Technical Problem

However, the conventional furan production methods described above require expensive metal catalysts such as Re, Ru, Ir, Pt, Pd, and Au, and in some cases more than one of the metal catalysts. Some reaction systems also require the use of pressurized O₂ or H₂ to advance to a state of equilibrium. Further, since most reactions must be carried out at a high temperature of 140 to 300° C., a high-temperature reactor such as an autoclave is required. These manufacturing constraints limit scale-up and contribute to significantly higher production costs.

In addition, the above conventional method of obtaining furoic acid from furfural tends to cause peroxidation of furfural, resulting in by-products (peroxides), and there is room for further improvement in terms of purity and yield.

### Solution to Problem

It is therefore an object of the present disclosure to provide simpler and less expensive methods for producing furoic acid derivatives, furan derivatives, phthalocyanine derivatives, and isoindoline derivatives, respectively.

Specifically, the present disclosure is as follows:
[1]A method for producing a furoic acid derivative, comprising an oxidation step of oxidizing a furfural derivative represented by general formula (1) to obtain a furoic acid derivative represented by general formula (2) as shown in reaction formula (I), wherein a pH of the reaction system is from 3 to 12,

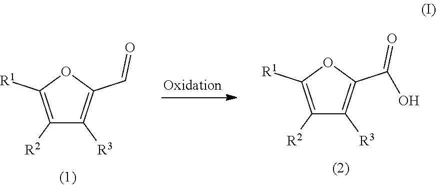

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
[2] The method for producing a furoic acid derivative according to [1], wherein one or more compounds selected from a group consisting of formic acid, hydrochloric acid, nitric acid, phosphoric acid, phosphoric anhydride, polyphosphoric acid, pyrophosphoric acid, sulfuric acid, methanesulfonic acid, p-toluenesulfonic acid, phosphorous acid, and salts thereof, and compounds represented by general formulas (A1) to (A4) are used as an additive in the oxidation step,

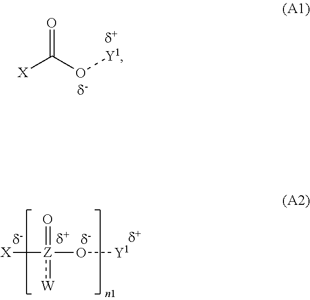

wherein,
X represents an —OH group, an —OM¹ group or an —R⁴,
wherein M¹ represents an alkali metal atom, and
R⁴ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Y¹ represents a hydrogen atom, an alkali metal atom, —COR⁵ or a functional group represented by general formula (B),
wherein R⁵ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Z represents a sulfur atom or a phosphorus atom,
W represents an oxygen atom or is roughly equivalent to the X,
n1 is an integer from 1 to 10,

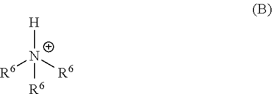

R⁶ in general formula (B) represents a hydrogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,

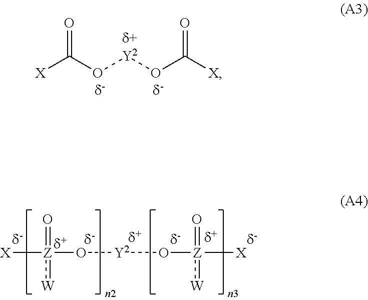

X in general formulas (A3) and (A4) each independently are roughly equivalent to the X in the general formulas (A1) and (A2),
Y² represents an alkaline earth metal atom,
   Z is roughly equivalent to the Z in the general formulas (A1) and (A2)
   W represents an oxygen atom or is roughly equivalent to the X in the general formulas (A1) and (A2), and
   n2 and n3 each, independently, is an integer from 1 to 10.
[3] The method for producing a furoic acid derivative according to [1] or [2], wherein O₂, H₂O₂, O₃, KMnO₄, KClO₃, or NaClO is used as an oxidant in the oxidation step.
[4] The method for producing a furoic acid derivative according to any one of [1] to [3], wherein the oxidation step is performed at a temperature of 0 to 120° C.
[5]A method for producing a furan derivative, comprising a decarboxylation step of decarboxylating a furoic acid derivative represented by general formula (2) to obtain a furan derivative represented by general formula (3) as shown in reaction formula (II), wherein decarboxylation is performed without a catalyst or using an oxide of mono- to tri-valent transition metal as a catalyst,

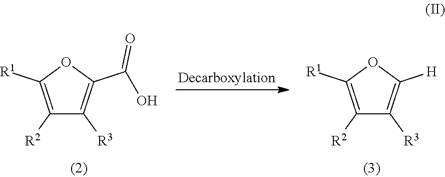

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
[6]A method for producing a furan derivative, comprising a decarboxylation step of decarboxylating a furoic acid derivative represented by general formula (2) to obtain a furan derivative represented by general formula (3) as shown in reaction formula (II), wherein decarboxylation is performed using a high boiling point solvent,

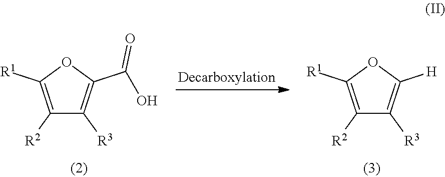

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
[7]A method for producing a furan derivative, comprising
   an oxidation step of oxidizing a furfural derivative represented by general formula (1) to obtain a furoic acid derivative represented by general formula (2) as shown in reaction formula (III), wherein a pH of the reaction system is from 3 to 12; and
   a decarboxylation step of decarboxylating the furoic acid derivative represented by the general formula (2) obtained in the oxidation step to obtain a furan derivative represented by general formula (3) as shown in reaction formula (III), wherein decarboxylation is performed without a catalyst or using an oxide of a mono- to tri-valent transition metal as a catalyst,

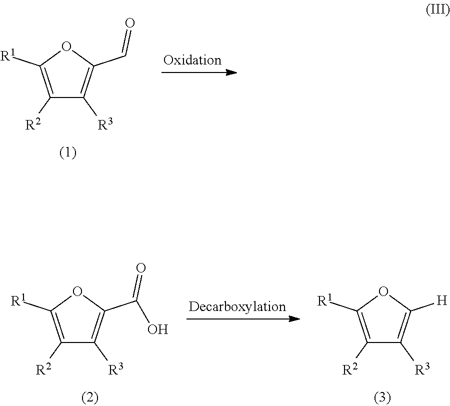

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
[8]A method for producing a furan derivative, comprising
   an oxidation step of oxidizing a furfural derivative represented by general formula (1) to obtain a furoic acid derivative represented by general formula (2) as shown in reaction formula (III), wherein a pH of the reaction system is from 3 to 12; and
   a decarboxylation step of decarboxylating the furoic acid derivative represented by the general formula (2) obtained in the oxidation step to obtain a furan derivative represented by general formula (3) as shown in reaction formula (III), wherein decarboxylation is performed using a high boiling point solvent.

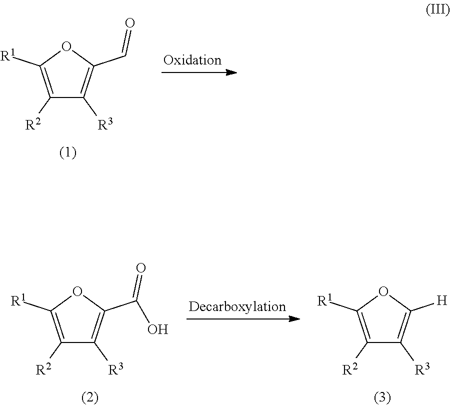

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
[9] The method for producing a furan derivative according to [7] or [8], wherein one or more compounds selected from a group consisting of formic acid, hydrochloric acid, nitric acid, phosphoric acid, phosphoric anhydride, polyphosphoric acid, pyrophosphoric acid, sulfuric acid, methanesulfonic acid, p-toluenesulfonic acid, phosphorous acid, and salts thereof, and compounds represented by general formulas (A1) to (A4) are used as an additive(s) in the oxidation step,

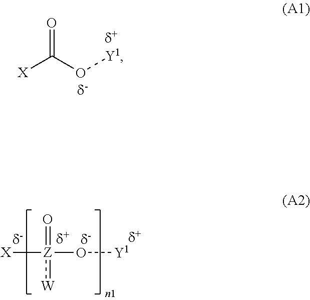

wherein,
X represents an —OH group, an —OM¹ group or an —R⁴,
wherein M¹ represents an alkali metal atom, and
R⁴ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Y¹ represents a hydrogen atom, an alkali metal atom, —COR⁵ or a functional group represented by general formula (B),
wherein R⁵ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Z represents a sulfur atom or a phosphorus atom,
W represents an oxygen atom or is roughly equivalent to the X,
n1 is an integer from 1 to 10,

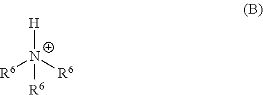

R⁶ in general formula (B) represents a hydrogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,

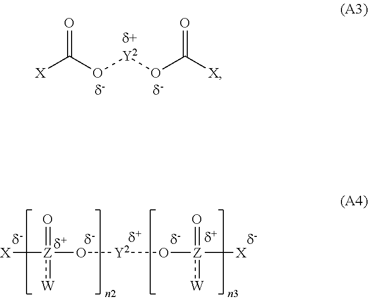

X in general formulas (A3) and (A4) each independently are roughly equivalent to the X in the general formulas (A1) and (A2),
Y² represents an alkaline earth metal atom,
Z is roughly equivalent to the Z in the general formulas (A1) and (A2)
W represents an oxygen atom or is roughly equivalent to the X in the general formulas (A1) and (A2), and
n2 and n3 each, independently, is an integer from 1 to 10.
[10] The method for producing a furan derivative according to any one of [7] to [9], wherein O₂, H₂O₂, O₃, KMnO₄, KClO₃, or NaClO is used as an oxidant in the oxidation step.
[11] The method for producing a furan derivative according to any one of [7] to [10], wherein the oxidation step is performed at a temperature from 0 to 120° C.
[12] The method for producing a furan derivative according to any one of [5] to [11], wherein a biomass degree of the furan derivative is 1% or more.
[13] A method for producing a phthalocyanine derivative from the furan derivative obtained by the method for producing a furan derivative according to any one of [5] to [12], comprising
   a step (A) of obtaining a compound represented by general formula (4) from the furan derivative represented by the general formula (3);
   a step (B) of obtaining a compound represented by general formula (5) from the compound represented by the general formula (4) obtained in the step (A); and
   a step (C) of obtaining a phthalocyanine derivative represented by general formula (6) or (7) from the compound represented by the general formula (5) obtained in the step (B),

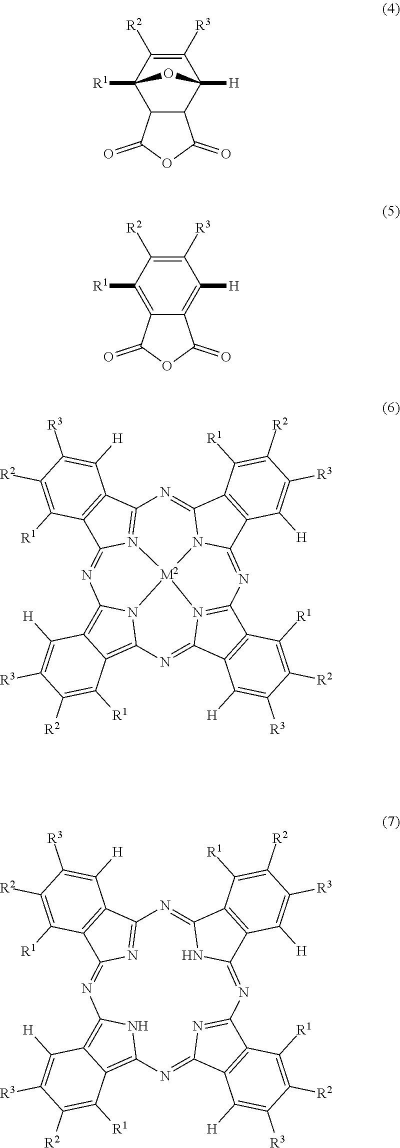

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
And M² represents a metal atom.
[14]A method for producing an isoindoline derivative from the furan derivative obtained by the method for producing a furan derivative according to any one of [5] to [12], comprising
   a step (a) of obtaining a compound represented by general formula (4) from the furan derivative represented by the general formula (3);
   a step (b) of obtaining a compound represented by general formula (5) from the compound represented by the general formula (4) obtained in the step (a); and
   a step (C) of obtaining at least one of isoindoline derivatives represented by general formulas (8) to (11) from the compound represented by the general formula (5) obtained in the step (b)

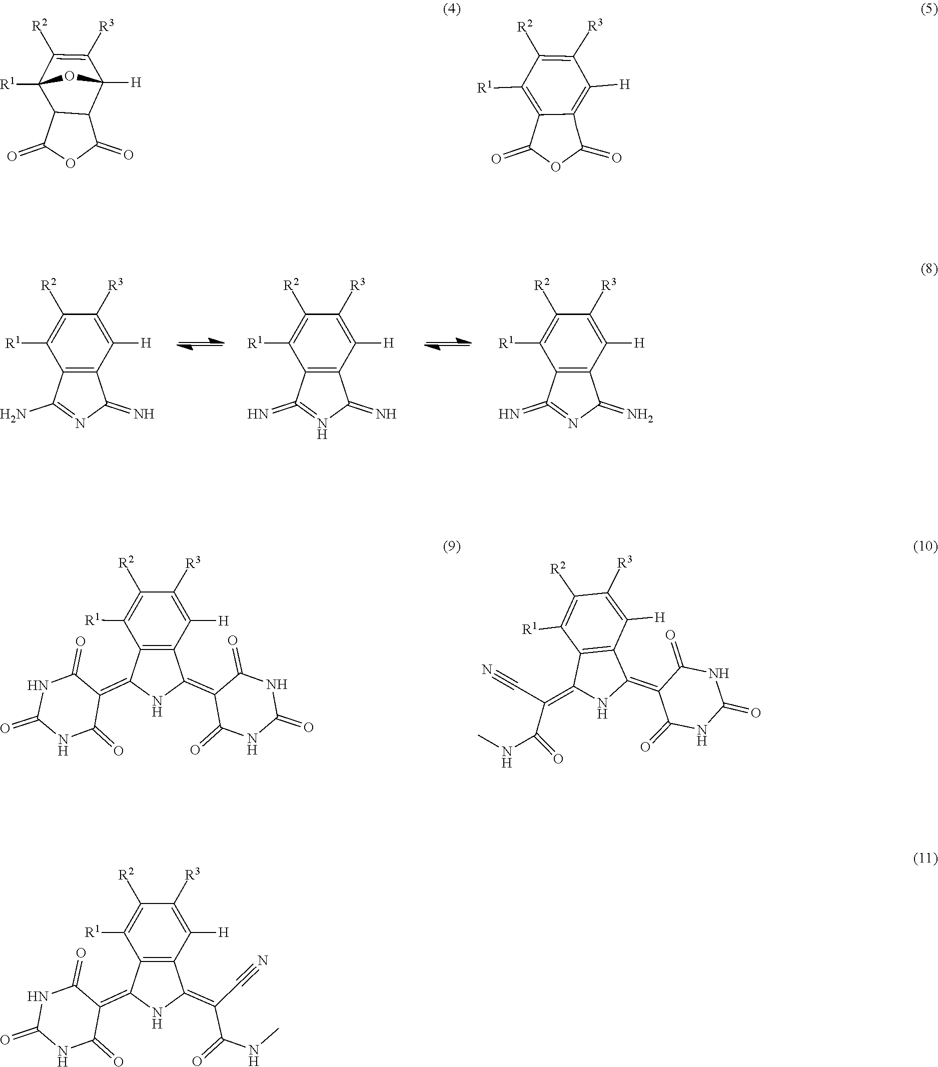

and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

### Advantageous Effect

According to the present disclosure, it is possible to provide simpler and less expensive methods for producing furoic acid derivatives, furan derivatives, phthalocyanine derivatives, and isoindoline derivatives, respectively.

### DETAILED DESCRIPTION

Certain embodiments of the present disclosure are described in detail below. The present disclosure is not limited to the following description, but may be expanded within the scope of the subject matter thereof.

As used herein, the term “roughly equivalent to” means not only “equivalent to” but also equivalent to the extent that the effect of the invention is not impaired.

### <Production Method of Furoic Acid Derivative>

A method for producing a furoic acid derivative of an embodiment may comprise an oxidation step of oxidizing a furfural derivative represented by general formula (1) to obtain a furoic acid derivative represented by general formula (2) as shown in reaction formula (I), wherein a pH of the reaction system is from 3 to 12.

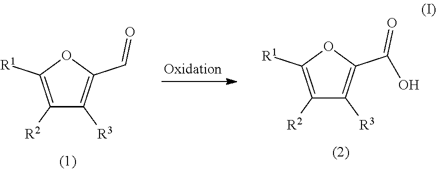

In the general formulas (1) and (2), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms. R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

The halogen atom may include, for example, a fluorine atom, a chlorine atom, a bromine atom, or an iodine atom.

As for the optionally substituted linear or branched alkyl group having 1 to 28 carbon atoms, the carbon number thereof is preferably 1 to 18, more preferably 1 to 12, even more preferably 1 to 6, and especially preferably 1 to 3. One or more (—CH₂-)s that are not adjacent to each other present in the alkyl group may be substituted with, for example, —C≡C—, —NH—, —N═, —CH═CH—, —O—, —S—, —COO—, —OCO—, or —CO—, and one or more hydrogen atoms present in the alkyl group may be substituted with, for example, a halogen atom such as a fluorine atom or a phenyl group.

As for the optionally substituted cycloalkyl group having 3 to 7 carbon atoms, the carbon number thereof is preferably 3 to 6, and more preferably 3 to 5. One or more (—CH₂-)s that are not adjacent to each other present in the cycloalkyl group may be substituted with, for example, —O—, —S—, —NH—, —N═, —COO—, —OCO—, or —CO—, and one or more hydrogen atoms present in the cycloalkyl group may be substituted with, for example, a halogen atom such as a fluorine atom or a phenyl group.

As for the optionally substituted aryl group having 6 to 12 carbon atoms, the carbon number thereof is preferably 6 to 10, and more preferably 6 to 9. One or more (—CH=)s that are not adjacent to each other present in the aryl group may be substituted with, for example, —N═, and one or more hydrogen atoms present in the aryl group may be substituted with, for example, a halogen atom such as a fluorine atom or a phenyl group.

R² and R³ may be ring-closed to form a five- to eight-membered ring. The ring structure may also have —OH group, —COOH group, —NH—, —S—, or —O— therein.

Specific examples of the furfural derivative represented by the general formula (1) include, but are not limited to, the following furfural derivatives (1-1) to (1-7).

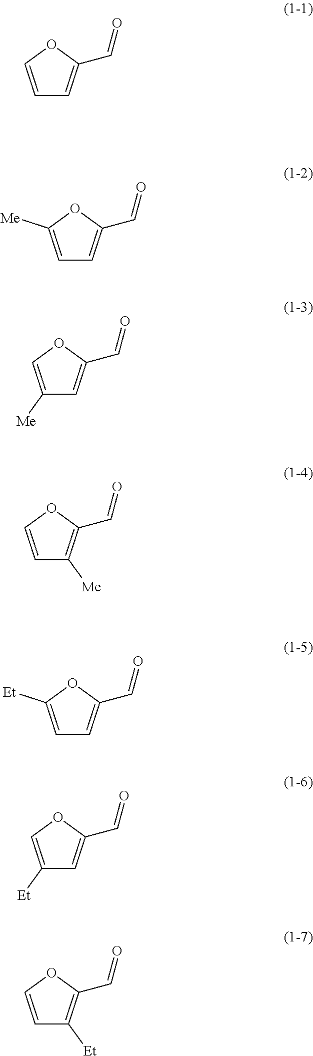

Specific examples of the furoic acid derivative represented by the general formula (2) include, but are not limited to, the following furoic acid derivatives (2-1) to (2-7).

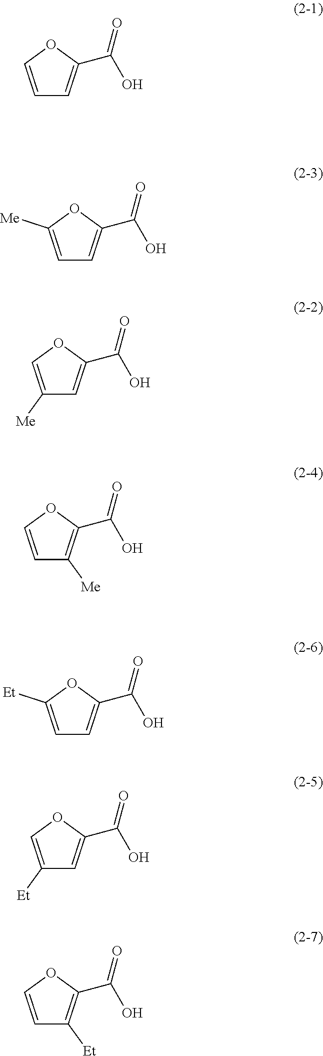

### [Oxidation Step]

In the oxidation step of the method for producing a furoic acid derivative of an embodiment, a furoic acid derivative represented by the general formula (2) can be obtained by the oxidation reaction of a furfural derivative represented by the general formula (1). In the oxidation reaction, the pH of the reaction system may be maintained within the range of 3 to 12 throughout the reaction. Generally, peroxidation tends to occur in the oxidation reaction of a furoic acid derivative. However, in the oxidation reaction of an embodiment, by controlling the pH of the reaction system within the range of 3 to 12, it is possible to suppress the production of peroxides (e.g., a furanone derivative, succinic acid derivative, a maleic acid derivative and a fumaric acid derivative) as a result of excessive oxidation, thus obtaining the furoic acid derivative in high purity and yields.

Although the pH of the reaction system varies as the oxidation reaction proceeds, it should preferably be in the range of 3 to 12 throughout the reaction. The lower limit of the pH is preferably 4 or higher, preferably 5 or higher, preferably 6 or higher, and preferably 7 or higher. The upper limit of the pH may be 11 or lower, or 10 or lower. Any single or combination of these upper and lower limits is acceptable.

Methods of controlling the pH of the reaction system in the above range include, for example, adding the additive(s) as described below.

The oxidizing agent used in the oxidation reaction is not limited as long as it allows the reaction to proceed suitably, but for example, O₂, H₂O₂, O₃, KMnO₄, KClO₃, or NaClO may be used. Among these oxidizing agents, H₂O₂ is preferred.

The amount of the oxidizing agent added is not limited as long as it is an amount that allows the reaction to proceed well, but the lower limit thereof is for example, preferably 50 mol % or more, more preferably 80 mol % or more, and even more preferably 100 mol % or more relative to the furfural derivative represented by the general formula (1). The upper limit is, for example, preferably 1000 mol % or less, more preferably 800 mol % or less, and even more preferably 400 mol % or less. Any single or combination of these upper and lower limits is acceptable.

In the oxidation reaction, one or more compounds selected from the group consisting of formic acid, hydrochloric acid, nitric acid, phosphoric acid, phosphoric anhydride, polyphosphoric acid, pyrophosphoric acid, sulfuric acid, methanesulfonic acid, p-toluenesulfonic acid, phosphorous acid, and salts thereof, and compounds represented by the following general formulas (A1) to (A4) are preferably used as an additive.

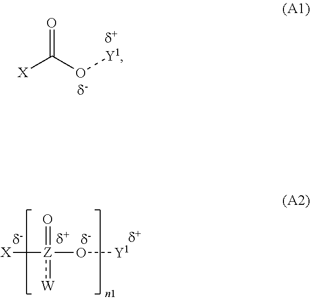

In the general formulas (A1) and (A2), X may represent an —OH group, an —OM¹ group or an —R⁴, wherein M¹ may represent an alkali metal atom, and R⁴ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms. Y¹ may represent a hydrogen atom, an alkali metal atom, —COR⁵ or a functional group represented by general formula (B), wherein R⁵ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms. Z may represent a sulfur atom or a phosphorus atom. W may represent an oxygen atom or may be roughly equivalent to the X. n1 may be an integer from 1 to 10.

The alkali metal atom of M¹ includes, for example, Li, Na or K.

The alkali metal atom of Y¹ includes, for example, Li, Na, K, Rb, or Cs.

Preferred ranges and examples of R⁴ and R⁵ may be the same as those listed above for the R¹ of the general formulas (1) and (2).

n1 is preferably an integer from 1 to 8, more preferably an integer from 1 to 5.

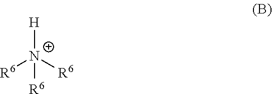

In the general formula (B), R⁶ may represent a hydrogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms.

Preferred ranges and examples of R⁶ may be the same as those listed above for the R¹ of the general formulas (1) and (2). Among these functional groups, protonated triethylamine is preferred.

X is particularly preferred to be —CH₃ group.

Y¹ is more preferably a hydrogen atom or an alkali metal atom, and even more preferably a hydrogen atom, Li, Na, K, Rb, or Cs.

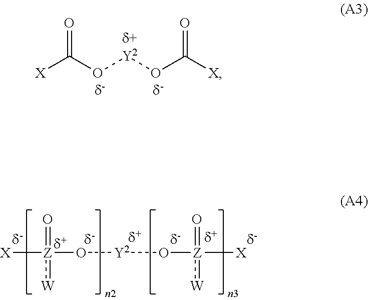

In the general formulas (A3) and (A4), X each independently may be roughly equivalent to the X in the general formulas (A1) and (A2). Y² may represent an alkaline earth metal atom. Z may be roughly equivalent to the Z in the general formulas (A1) and (A2). W may represent an oxygen atom or may be roughly equivalent to the X in the general formulas (A1) and (A2). n2 and n3 each, independently, may be an integer from 1 to 10.

The alkali earth metal atom of Y² may include, for example, Be, Mg, Ca, Sr, or Ba.

n2 is preferably an integer from 1 to 8, more preferably an integer from 1 to 5. n3 is preferably an integer from 1 to 8, more preferably an integer from 1 to 5.

The additive is more preferably one or more compounds selected from the group consisting of phosphates and compounds represented by the following general formulas (A1) to (A4), and even more preferably one or more compounds selected from the group consisting of NaH₂PO₄, potassium acetate, sodium acetate, and triethylammonium acetate.

In the oxidation reaction of a furfural derivative, the pH of the reaction system may become lower (i.e. shifts to the acidic side) as the reaction proceeds, and peroxides are more likely to be generated, and the generated peroxides act as autocatalysts. However, the addition of additive(s) may neutralize the reaction system, making it easier to maintain the pH between 3 and 12 and to obtain the furoic acid derivatives in higher purity and yields.

As for the amount of the additive added, the lower limit thereof is preferably 50 mol % or more, preferably 70 mol % or more, and preferably 100 mol % or more relative to the furfural derivative represented by the general formula (1). The upper limit is preferably 500 mol % or less, preferably 300 mol % or less, and preferably 200 mol % or less. Any single or combination of these upper and lower limits is acceptable.

The timing for adding the additive is not limited as long as the pH of the reaction system can be maintained in the range of 3 to 12. The total amount of the additive may be added at any time before or during the reaction. Additionally, additive(s) may be added several times before or during the reaction.

The solvent used in the oxidation reaction is not limited as long as it allows the reaction to proceed suitably, but for example, water, 1,4-dioxane, isobutyl methyl ketone, toluene, xylene, alkyl benzene, dimethyl sulfoxide, N,N-dimethylformamide, acetonitrile, ethanol, methanol, isopropanol, tert-amyl alcohol, or ethyl acetate may be used. Additionally, the oxidation reaction may also proceed without a solvent.

The temperature of the oxidation reaction is preferably 0 to 120° C., more preferably 5 to 100° C., and even more preferably 30 to 90° C. The oxidation reaction in an embodiment can be performed at lower temperatures than in conventional technologies. Conventional technologies appear to require reactions at a high temperature (140 to 300° C.). Certain embodiments eliminate the need for a high-temperature reactor, thus reducing production costs and allowing for easy scale-up.

The time of the oxidation reaction is not limited and may be set according to the reaction temperature and the types of the additive(s) and the oxidizing agent.

The method for producing a furoic acid derivative of an embodiment may include other steps, such as a purification step of removing reaction byproducts.

Since the method for producing a furoic acid derivative of an embodiment is simple and inexpensive, it can be performed using ordinary laboratory facilities and can be easily scaled up. In addition, since the reaction may be carried out under relatively mild reaction conditions, the reaction may be easy to control, and the formation of byproducts (peroxides) due to peroxidation may be low.

According to the method for producing a furoic acid derivative of an embodiment, high purity furoic acid derivatives can be obtained. As for the purity (content) of the furoic acid derivative, the lower limit thereof is preferably 95.0% or more, more preferably 98.0% or more, and even more preferably 99.0% or more. The upper limit is not limited, and while 100% is most preferred, it may be 99.999% or less, or 99.5% or less. Any single or combination of these upper and lower limits is acceptable.

As for the content of the reaction byproducts such as peroxides, the upper limit is preferably 5.0% or less, more preferably 2.0% or less, and even more preferably 1.0% or less. The lower limit is not limited, and while 0% is most preferred, it may be 0.01% or more, or 0.1% or more. Any single or combination of these upper and lower limits is acceptable.

The purity (content) of the furoic acid derivative and the content of the reaction byproducts can be determined, for example, by quantifying the furoic acid and the reaction byproducts, respectively, using gas chromatography-mass spectrometry (GC-MS).

According to the method for producing a furoic acid derivative of an embodiment, furoic acid derivatives can be obtained in a high yield. As for the yield of the furoic acid derivative, the lower limit thereof is preferably 10% or more, more preferably 50% or more, and even more preferably 80% or more. The upper limit is not limited, and while 100% is most preferred, it may be 95% or less, or 90% or less. Any single or combination of these upper and lower limits is acceptable.

### <Production Method of Furan Derivative>

A method for producing a furan derivative of an embodiment may comprise a decarboxylation step of decarboxylating a furoic acid derivative represented by general formula (2) to obtain a furan derivative represented by general formula (3) as shown in reaction formula (II), wherein decarboxylation is performed without a catalyst or using an oxide of mono- to tri-valent transition metal as a catalyst.

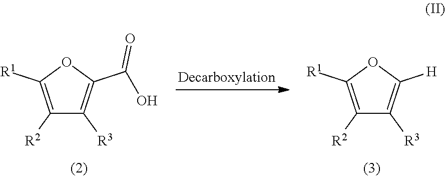

In the general formulas (2) and (3), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

Preferred ranges and examples of R¹ to R³ may be the same as those listed above for the R¹ to R³ of the general formulas (1) and (2) in the method for producing a furoic acid derivative of an embodiment.

Specific examples of the furoic acid derivative represented by the general formula (2) may be the same as those listed above in the method for producing a furoic acid derivative of an embodiment.

Specific examples of the furan derivative represented by the general formula (3) include, but are not limited to, the following furfural derivatives (3-1) to (3-7).

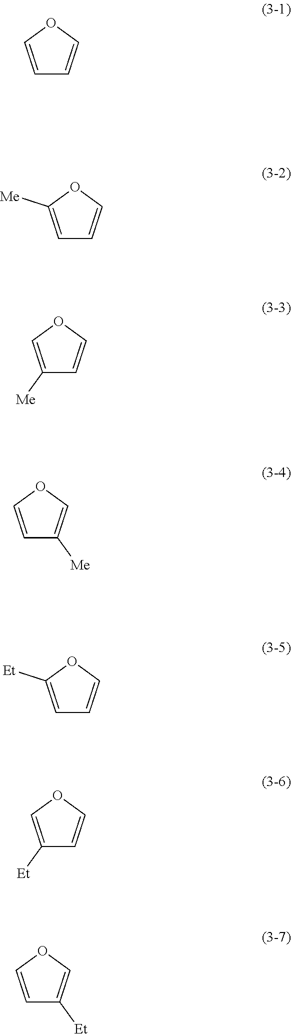

### <Decarboxylation Step>

In the decarboxylation step of a method for producing a furan derivative of an embodiment, a furan derivative represented by the general formula (3) can be obtained by the decarboxylation reaction of a furoic acid derivative represented by the general formula (2). Unlike conventional technologies that use expensive and valuable metal catalysts (e.g., Re, Ru, Ir, Pt, Pd, Au), the decarboxylation reaction can be performed without a catalyst or using an inexpensive and easily available oxide of mono- to tri-valent transition metal as a catalyst, making scale-up easy and reducing production costs.

When the decarboxylation reaction is performed without a catalyst, the reaction temperature must be relatively high in order to promote good decarboxylation. Therefore, it is more preferred to use an oxide of mono- to tri-valent transition metal as a catalyst.

When a catalyst is used in the decarboxylation reaction, specific examples of the catalyst include Cu₂O, CuO, FeO, and Fe₂O₃. Among these catalysts, Cu₂O is preferred.

The amount of the catalyst added is not limited as long as it is an amount that allows the reaction to proceed well, but the lower limit thereof is for example, preferably 0.001 mol % or more, more preferably 0.01 mol % or more, and even more preferably 0.1 mol % or more relative to the furoic acid derivative represented by the general formula (2). The upper limit is, for example, preferably 100 mol % or less, more preferably 50 mol % or less, and even more preferably 20 mol % or less. Any single or combination of these upper and lower limits is acceptable.

The solvent used in the decarboxylation reaction is not limited as long as it allows the reaction to proceed suitably. From the viewpoint of suppressing the sublimation of the furan derivative that occurs when the reaction temperature is high, the solvent is preferably a high boiling point solvent, for example, dimethylacetamide (DMA), N-methyl-2-pyrrolidone (NMP), N-ethyl-2-pyrrolidone (NEP), dimethyl sulfoxide (DMSO), dimethylformamide (DMF), dodecyl alcohol, diethylene glycol monobutyl ether, hexadecane, or alkyl benzene. Among these solvents, N-methyl-2-pyrrolidone, N-ethyl-2-pyrrolidone, or hexadecane is preferred.

A high boiling point solvent may generally refer to a solvent with a boiling point of 150 to 280° C. However, other boiling points may also be considered a high boiling point, particularly any boiling point above the earlier recited range.

The decarboxylation reaction also proceeds even without a solvent.

The temperature of the decarboxylation reaction is not limited as long as it allows the reaction to proceed suitably, but for example, when the reaction is performed without a catalyst, it is preferably 50 to 300° C., more preferably 80 to 290° C., and even more preferably 100 to 280° C. When an oxide of mono- to tri-valent transition metal is used as a catalyst, it is preferably 50 to 280° C., more preferably 80 to 270° C., and even more preferably 100 to 250° C.

The time of the decarboxylation reaction is not limited and may be set according to the reaction temperature and the type of the catalyst.

A method for producing a furan derivative of an embodiment may comprise a decarboxylation step of decarboxylating a furoic acid derivative represented by the following general formula (2) to obtain a furan derivative represented by the following general formula (3), wherein decarboxylation is performed using a high boiling point solvent as shown in the following reaction formula (II).

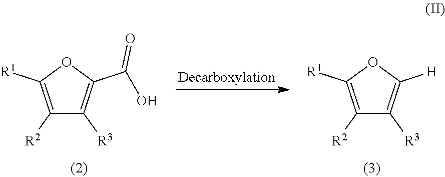

In the general formulas (2) and (3), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

Preferred ranges and examples of R¹ to R³ may be the same as those listed above for the R¹ to R³ of the general formulas (1) and (2) in the method for producing a furoic acid derivative of an embodiment.

Specific examples of the furoic acid derivative represented by the general formula (2) are the same as those listed above in the method for producing a furoic acid derivative of an embodiment.

Specific examples of the furan derivative represented by the general formula (3) are the same as those listed above.

### <Decarboxylation Step>

In the decarboxylation step of a method for producing a furan derivative of an embodiment, a furan derivative represented by the general formula (3) can be obtained by the decarboxylation reaction of a furoic acid derivative represented by the general formula (2). In decarboxylation reactions, high reaction temperatures can cause the undesired sublimation of furan derivatives. Therefore, catalysts are typically used in order to lower reaction temperatures and assist in the prevention of sublimation. In the decarboxylation reaction of an embodiment, through the use of high boiling point solvents, such decarboxylation reactions can successfully proceed at higher reaction temperatures and without the undesired sublimation of furan derivatives. In such a manner, decarboxylation reactions can successfully occur with or without the use of catalysts.

The specific examples of the high boiling solvents are the same as those listed above.

The decarboxylation reaction may be performed with or without a catalyst. When a catalyst is used in the decarboxylation reaction, the catalyst is not limited as long as it allows the reaction to proceed suitably. From the viewpoint of ease of scale-up and reduction of production costs, the catalyst is preferably an oxide of a mono- to tri-valent transition metal. Specific examples of the oxide of a mono- to tri-valent transition metal are the same as those listed above.

The amount of the catalyst added is not limited as long as it is an amount that allows the reaction to proceed well, but the lower limit thereof is for example, preferably 0.001 mol % or more, more preferably 0.01 mol % or more, and even more preferably 0.1 mol % or more relative to the furoic acid derivative represented by the general formula (2). The upper limit is, for example, preferably 100 mol % or less, more preferably 50 mol % or less, and even more preferably 20 mol % or less. Any single or combination of these upper and lower limits is acceptable.

The temperature of the decarboxylation reaction is not limited as long as it allows the reaction to proceed suitably, but for example, when the reaction is performed without a catalyst, it is preferably 50 to 300° C., more preferably 80 to 290° C., and even more preferably 100 to 280° C. When an oxide of mono- to tri-valent transition metal is used as a catalyst, it is preferably 50 to 280° C., more preferably 80 to 270° C., and even more preferably 100 to 250° C.

The time of the decarboxylation reaction is not limited and may be set according to the reaction temperature and the type of the catalyst.

Another method for producing a furan derivative of an embodiment may comprise an oxidation step of oxidizing a furfural derivative represented by general formula (1) to obtain a furoic acid derivative represented by general formula (2) as shown in reaction formula (III), wherein a pH of the reaction system is from 3 to 12; and
a decarboxylation step of decarboxylating the furoic acid derivative represented by the general formula (2) obtained in the oxidation step to obtain a furan derivative represented by general formula (3) as shown in reaction formula (III), wherein decarboxylation is performed without a catalyst or using an oxide of a mono- to tri-valent transition metal as a catalyst,

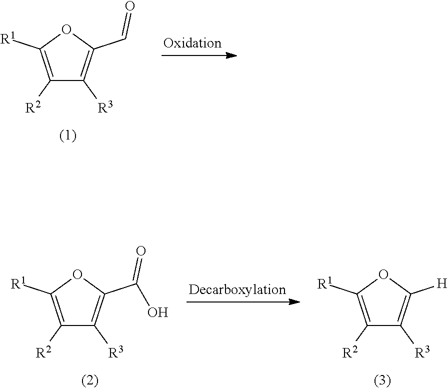

(III)

In the general formulas (1) to (3), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

Preferred ranges and examples of R¹ to R³ may be the same as those listed above for the R¹ to R³ of the general formulas (1) and (2) in the method for producing a furoic acid derivative of an embodiment.

Specific examples of the furfural derivative represented by the following general formula (1), the furoic acid derivative represented by the general formula (2), and the furan derivative represented by the general formula (3) are the same as those listed above.

### [Oxidation Step]

In the oxidation step a method for producing a furan derivative of an embodiment, a furoic acid derivative represented by the general formula (2) can be obtained by the oxidation reaction of a furfural derivative represented by the general formula (1). In the oxidation reaction, the pH of the reaction system may be maintained within the range of 3 to 12 throughout the reaction. Generally, peroxidation tends to occur in the oxidation reaction of a furoic acid derivative. However, in the oxidation reaction of an embodiment, by controlling the pH of the reaction system within the range of 3 to 12, it is possible to suppress the production of peroxides (e.g., a succinic acid derivative, a furanone derivative, maleic acid derivative and a fumaric acid derivative) as a result of excessive oxidation, thus obtaining the furoic acid derivative in high purity and yields.

The preferred ranges of the pH of the reaction system may be the same as those listed above for the oxidation step of the method for producing a furoic acid derivative.

Methods of controlling the pH of the reaction system in the above range include, for example, adding an additive as described above for the oxidation step of the method for producing a furoic acid derivative.

In the oxidation reaction, the oxidizing agents, the additives, and the solvents listed above for the oxidation step of the method for producing a furoic acid derivative can be used.

The timing for adding the additive may be the same as in the oxidation step of the method for producing a furoic acid derivative.

The temperature of the oxidation reaction can be the same as the temperature of the oxidation step of the method for producing a furoic acid derivative. The oxidation reaction in an embodiment can be performed at lower temperatures than in conventional technologies. Conventional technologies appear to require reactions at a high temperature (140 to 300° C.). Certain embodiments eliminate the need for a high-temperature reactor, thus reducing production costs and allowing for easy scale-up.

The time of the oxidation reaction is not limited and may be set according to the reaction temperature and the types of the additive(s) and the oxidizing agent.

### <Decarboxylation Step>

In the decarboxylation step of a method for producing a furan derivative of an embodiment, a furan derivative represented by the general formula (3) can be obtained by the decarboxylation reaction of a furoic acid derivative represented by the general formula (2).

Since the decarboxylation reaction may use as raw material the furoic acid derivative represented by the general formula (2) obtained in high purity and high yield by the above oxidation step, the furan derivative can be obtained in high purity and high yield.

Unlike conventional technologies that use expensive and valuable metal catalysts (e.g., Re, Ru, Ir, Pt, Pd, Au), the decarboxylation reaction can be performed without a catalyst or using an inexpensive and easily available oxide of mono- to tri-valent transition metal as a catalyst, making scale-up easy and reducing production costs.

When the decarboxylation reaction is performed without a catalyst, the reaction temperature is typically relatively high in order to promote good decarboxylation. Therefore, it is more preferred to use an oxide of mono- to tri-valent transition metal as a catalyst.

In the decarboxylation reaction, the catalysts and solvents listed above can be used.

The temperature of the decarboxylation reaction can be the same temperature as in embodiments recited above.

The time of the decarboxylation reaction is not limited and may be set according to the reaction temperature and the type of the catalyst.

In the method for producing a furan derivative of an embodiment, the first-stage oxidation step may be carried out under relatively mild reaction conditions, which makes the reaction easy to control and may reduce the formation of byproducts (peroxides) due to peroxidation.

Since the second-stage decarboxylation process may be performed using the furoic acid derivative obtained in high purity and high yield by the first-stage oxidation step, the furan derivative can be produced in high purity and high yield. In addition, the reaction can be performed without a catalyst or with an inexpensive and easily available catalyst.

Therefore, a method for producing a furan derivative of an embodiment is a two-step process, but it is simple and inexpensive. It can be performed using ordinary laboratory facilities, facilitating scale-up and reducing manufacturing costs.

Another method for producing a furan derivative of an embodiment may comprise an oxidation step of oxidizing a furfural derivative represented by general formula (1) to obtain a furoic acid derivative represented by general formula (2) as shown in reaction formula (III), wherein a pH of the reaction system is from 3 to 12; and
a decarboxylation step of decarboxylating the furoic acid derivative represented by the general formula (2) obtained in the oxidation step to obtain a furan derivative represented by general formula (3) as shown in reaction formula (III), wherein decarboxylation is performed using a high boiling point solvent.

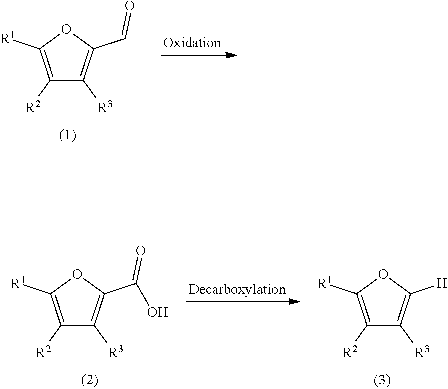

(III)

In the general formulas (1) to (3), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

Preferred ranges and examples of R¹ to R³ may be the same as those listed above for the R¹ to R³ of the general formulas (1) and (2) in the method for producing a furoic acid derivative of an embodiment.

Specific examples of the furfural derivative represented by the following general formula (1), the furoic acid derivative represented by the general formula (2), and the furan derivative represented by the general formula (3) are the same as those listed above.

### [Oxidation Step]

In the oxidation step of a method for producing a furan derivative of an embodiment, a furoic acid derivative represented by the general formula (2) can be obtained by the oxidation reaction of a furfural derivative represented by the general formula (1). In the oxidation reaction, the pH of the reaction system may be maintained within the range of 3 to 12 throughout the reaction. Generally, peroxidation tends to occur in the oxidation reaction of a furoic acid derivative. However, in the oxidation reaction of an embodiment, by controlling the pH of the reaction system within the range of 3 to 12, it is possible to suppress the production of peroxides (e.g., a succinic acid derivative, a furanone derivative, a maleic acid derivative and a fumaric acid derivative) as a result of excessive oxidation, thus obtaining the furoic acid derivative in high purity and yields.

The preferred ranges of the pH of the reaction system may be the same as those listed above for the oxidation step of the method for producing a furoic acid derivative.

Methods of controlling the pH of the reaction system in the above range include, for example, adding an additive as described above for the oxidation step of the method for producing a furoic acid derivative.

In the oxidation reaction, the oxidizing agents, the additives, and the solvents listed above for the oxidation step of the method for producing a furoic acid derivative can be used.

The timing for adding the additive may be the same as in the oxidation step of the method for producing a furoic acid derivative.

The temperature of the oxidation reaction can be the same as the temperature of the oxidation step of the method for producing a furoic acid derivative. The oxidation reaction in an embodiment can be performed at lower temperatures than in conventional technologies. Conventional technologies appear to require reactions at a high temperature (140 to 300° C.). Certain embodiments eliminate the need for a high-temperature reactor, thus reducing production costs and allowing for easy scale-up.

The time of the oxidation reaction is not limited and may be set according to the reaction temperature and the types of the additive(s) and the oxidizing agent.

### <Decarboxylation Step>

In the decarboxylation step of a method for producing a furan derivative of an embodiment, a furan derivative represented by the general formula (3) can be obtained by the decarboxylation reaction of a furoic acid derivative represented by the general formula (2). In decarboxylation reactions, high reaction temperatures can cause the undesired sublimation of furan derivatives. Therefore, catalysts are typically used in order to lower reaction temperatures and assist in the prevention of sublimation. In the decarboxylation reaction of an embodiment, through the use of high boiling point solvents, such decarboxylation reactions can successfully proceed at higher reaction temperatures and without the undesired sublimation of furan derivatives. In such a manner, decarboxylation reactions can successfully occur with or without the use of catalysts.

The specific examples of the high boiling solvents may be the same as those listed above.

The decarboxylation reaction may be performed without a catalyst or with a catalyst. The specific examples of the catalysts may be the same as those listed above.

The temperature of the decarboxylation reaction can be the same as temperatures listed above.

The time of the decarboxylation reaction is not limited and may be set according to the reaction temperature and the type of the catalyst.

In the method for producing a furan derivative of an embodiment, the first-stage oxidation step may be carried out under relatively mild reaction conditions, which makes the reaction easy to control and may reduce the formation of byproducts (peroxides) due to peroxidation.

Since the second-stage decarboxylation process may be performed in a high boiling point solvent and the furoic acid derivative obtained in high purity and high yield by the first-stage oxidation step, the furan derivative can be produced in high purity and high yield.

Therefore, a method for producing a furan derivative of an embodiment is a two-step process, but it is simple and inexpensive. It can be performed using ordinary laboratory facilities, facilitating scale-up and reducing manufacturing costs.

Each method for producing a furan derivative of an embodiment may include steps other than the oxidation step and the decarboxylation step. For example, steps such as a purification step of removing reaction byproducts step may be included.

According to methods for producing a furan derivative of an embodiment, high purity furan derivatives can be obtained. As for the purity (content) of the furan derivative, the lower limit thereof is preferably 99.5% or more, more preferably 99.6% or more, and even more preferably 99.7% or more. The upper limit is not limited, and while 100% is most preferred, it may be 99.999% or less, or 99.98% or less. Any single or combination of these upper and lower limits is acceptable.

As for the content of the reaction byproducts, the upper limit is preferably 0.5% or less, more preferably 0.3% or less, and even more preferably 0.2% or less. The lower limit is not limited, and while 0% is most preferred, it may be 0.001% or more, or 0.01% or more. Any single or combination of these upper and lower limits is acceptable.

The purity (content) of the furan derivative and the content of the reaction byproducts can be determined, for example, by quantifying the furoic acid and the reaction byproducts, respectively, using gas chromatography-mass spectrometry (GC-MS).

According to methods for producing a furan derivative of an embodiment, furan derivatives can be obtained in a high yield. As for the yield of the furan derivative, the lower limit thereof is preferably 99.5% or more, more preferably 99.6% or more, and even more preferably 99.7% or more. The upper limit is not limited, and while 100% is most preferred, it may be 99.99% or less, or 99.98% or less. Any single or combination of these upper and lower limits is acceptable.

### <Production Methods of Phthalocyanine Derivative>

A method for producing a phthalocyanine derivative of an embodiment may use the furan derivative represented by the general formula (3), which may be obtained by the method for producing a furan derivative of an embodiment.

A method for producing a phthalocyanine derivative of an embodiment may comprise a step (A) of obtaining a compound represented by general formula (4) from the furan derivative represented by the general formula (3); a step (B) of obtaining a compound represented by general formula (5) from the compound represented by the general formula (4) obtained in the step (A); and a step (C) of obtaining a phthalocyanine derivative represented by general formula (6) or (7) from the compound represented by the general formula (5) obtained in the step (B).

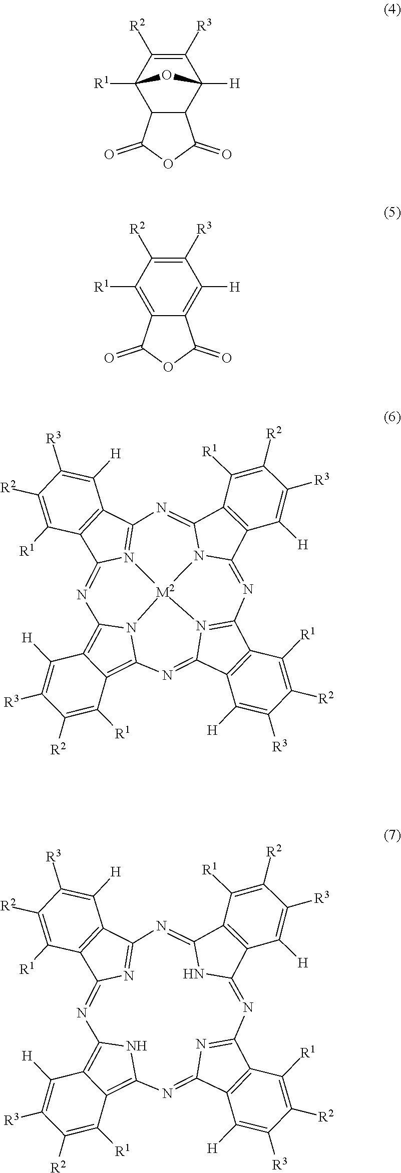

In the general formulas (4) to (7), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

In the general formula (6), M² may represent a metal atom.

Preferred ranges and examples of R¹ to R³ may be the same as those listed above for the R¹ to R³ of the general formulas (1) and (2) in the method for producing a furoic acid derivative of an embodiment.

### [Step (A)]

In the step (A), a furan derivative represented by the general formula (3) and maleic anhydride are subjected to the Diels-Alder reaction to produce a compound represented by the general formula (4) (a DA intermediate).

Specific examples of the furan derivative represented by the general formula (3) are the same as those listed above.

Specific examples of the compound represented by the general formula (4) (the DA intermediate) include, but are not limited to, the following compounds (4-1) to (4-11).

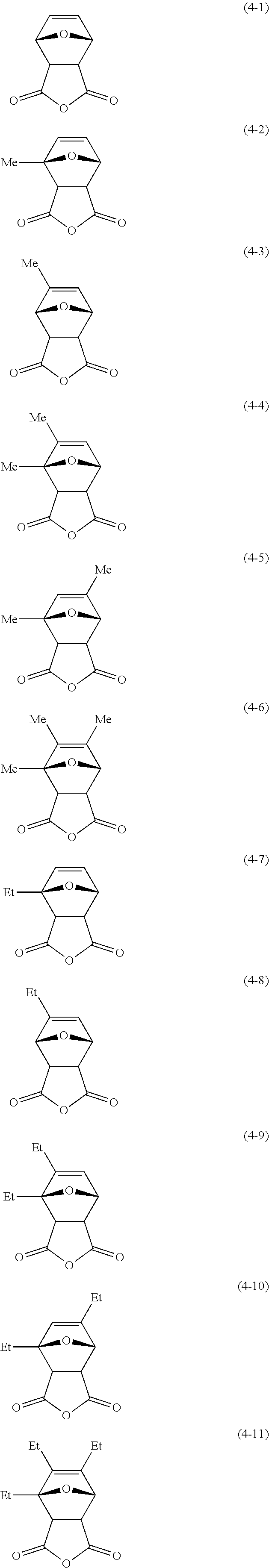

The reaction solvent is not limited as long as it allows the reaction to proceed suitably, but chloroform, dioxane, ethyl acetate, alkyl benzene, toluene, xylene, or diethyl ether are preferred.

The reaction temperature is not limited as long as it allows the reaction to proceed suitably, but the lower limit is preferably −10° C. or higher, more preferably 0° C. or higher, even more preferably 10° C. or higher, and especially preferably 15° C. or higher. The upper limit is preferably 100° C. or lower, more preferably 80° C. or lower, even more preferably 70° C. or lower, and especially preferably 50° C. or lower. Any single or combination of these upper and lower limits is acceptable.

The reaction pressure is not limited as long as it allows the reaction to proceed suitably, but the lower limit is preferably 0.1 Mpa or higher, preferably 0.2 Mpa or higher, preferably 0.3 Mpa or higher, and preferably 0.4 Mpa or higher. The upper limit is preferably 5 Mpa or lower, preferably 3 Mpa or lower, preferably 1 Mpa or lower, preferably 0.9 Mpa or lower, preferably 0.8 Mpa or lower, preferably 0.7 Mpa or lower, preferably 0.6 Mpa or lower, and preferably 0.5 Mpa or lower. Any single or combination of these upper and lower limits is acceptable.

### [Step (B)]

In the step (B), by ring-opening dehydration of the compound represented by the general formula (4) (the DA intermediate) obtained in the step (A), a compound represented by the general formula (5) (a phthalic anhydride derivative) can be produced.

Specific examples of the compound represented by the general formula (5) (the phthalic anhydride derivative) include, but are not limited to, the following compounds (5-1) to (5-11).

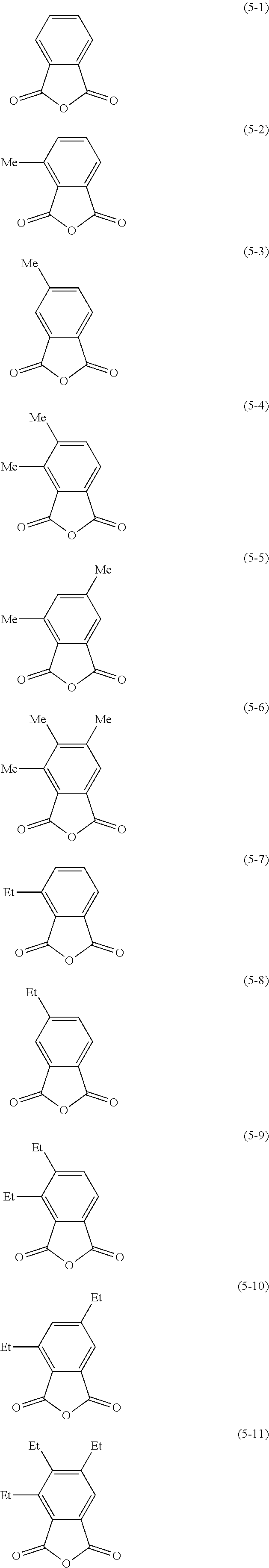

The reaction solvent is not limited as long as it allows the reaction to proceed suitably, but water, acetonitrile, toluene, xylene, alkyl benzene, or a mixture thereof is preferred. The ring-opening dehydration reaction may also be carried out without a solvent.

The reaction temperature is not limited as long as it allows the reaction to proceed suitably, but the lower limit is preferably 20° C. or higher, preferably 25° C. or higher, preferably 30° C. or higher, preferably 35° C. or higher, and preferably 40° C. or higher. The upper limit is preferably 150° C. or lower, preferably 140° C. or lower, preferably 130° C. or lower, preferably 120° C. or lower, preferably 110° C. or lower, and preferably 100° C. or lower. Any single or combination of these upper and lower limits is acceptable.

For the ring-opening dehydration reaction, the use of a catalyst is preferred. As a catalyst, the same compounds listed above as the additives in the oxidation step of the method for producing a furoic acid derivatives of an embodiment can be used. Other compounds may also be used.

As for the amount of the catalyst added, the lower limit thereof is preferably 0.1 mol % or more, preferably 0.5 mol % or more, preferably 1 mol % or more, preferably 5 mol % or more, preferably 10 mol % or more, preferably 20 mol % or more, preferably 50 mol % or more, preferably 70 mol % or more, preferably 100 mol % or more, preferably 150 mol % or more, preferably 200 mol % or more, preferably 250 mol % or more, and preferably 300 mol % or more relative to the compound represented by the general formula (4) (the DA intermediate). The upper limit is preferably 3000 mol % or less, preferably 2500 mol % or less, preferably 2000 mol % or less, preferably 1500 mol % or less, preferably 1000 mol % or less, and preferably 500 mol % or less. Any single or combination of these upper and lower limits is acceptable.

### [Step (C)]

In the step (C), by ring-opening dehydration of the compound represented by the general formula (5) (the phthalic anhydride derivative) obtained in the step (B) is reacted with urea and M²X (M² is a metal atom and X is a halogen atom) in the presence of a catalyst to produce a phthalocyanine derivative represented by the general formula (6).

Further, a phthalocyanine derivative represented by the general formula (7) can be produced by performing a demetalization reaction on the obtained phthalocyanine derivative represented by the general formula (6).

The reaction solvent is not limited as long as it allows the reaction to proceed suitably, but alkyl benzene is preferred. The reaction may also be carried out without a solvent.

The reaction temperature is not limited as long as it allows the reaction to proceed suitably, but the lower limit is preferably 100° C. or higher, preferably 110° C. or higher, preferably 120° C. or higher, preferably 130° C. or higher, preferably 140° C. or higher, and preferably 150° C. or higher. The upper limit is preferably 250° C. or lower, preferably 240° C. or lower, preferably 230° C. or lower, preferably 220° C. or lower, preferably 210° C. or lower, and preferably 200° C. or lower. Any single or combination of these upper and lower limits is acceptable.

M² of M²X may represent a metal atom. For example, it is preferably A1, Si, Sc, Ti, V, Mg, Fe, Co, Ni, Cu, Zn, Ga, Ge, Y, Zr, Nb, In, Sn, or Pb, and more preferably A1, Fe, Cu, or Zn, and even more preferably Cu or Zn.

X of M²X may represent a halogen atom. For example, it is preferably a chlorine atom.

The catalyst is not limited as long as it allows the reaction to proceed suitably, but a molybdenum catalyst is preferred, and ammonium molybdate (VI) tetrahydrate is more preferred.

As for the amount of the catalyst added, the lower limit thereof is preferably 0.001 mol % or more, more preferably 0.01 mol % or more, and even more preferably 0.1 mol % or more relative to the compound represented by the general formula (5) (the phthalic anhydride derivative). The upper limit thereof is preferably 10 mol % or less, more preferably 5 mol % or less, and even more preferably 3 mol % or less. Any single or combination of these upper and lower limits is acceptable.

The demetalization reaction of the phthalocyanine derivative represented by the general formula (6) includes, but is not limited to, for example, the method described in Chemical Communication, 2009, 1970-1971.

Since the raw material, a furan derivative, may be produced using a method for producing a furan derivative of an embodiment, the method for producing a phthalocyanine derivative of an embodiment may be simple and inexpensive and can be performed using ordinary laboratory facilities, facilitating scale-up and reducing manufacturing costs.

### <Production Methods of Isoindoline Derivative>

A method for producing an isoindoline derivative of an embodiment may use the furan derivative represented by the general formula (3), which may be obtained by the method for producing a furan derivative of an embodiment.

A method for producing an isoindoline derivative of an embodiment may comprise a step (a) of obtaining a compound represented by general formula (4) from the furan derivative represented by the general formula (3); a step (b) of obtaining a compound represented by general formula (5) from the compound represented by the general formula (4) obtained in the step (a); and a step (C) of obtaining at least one of isoindoline derivatives represented by general formulas (8) to (11) from the compound represented by the general formula (5) obtained in the step (b).

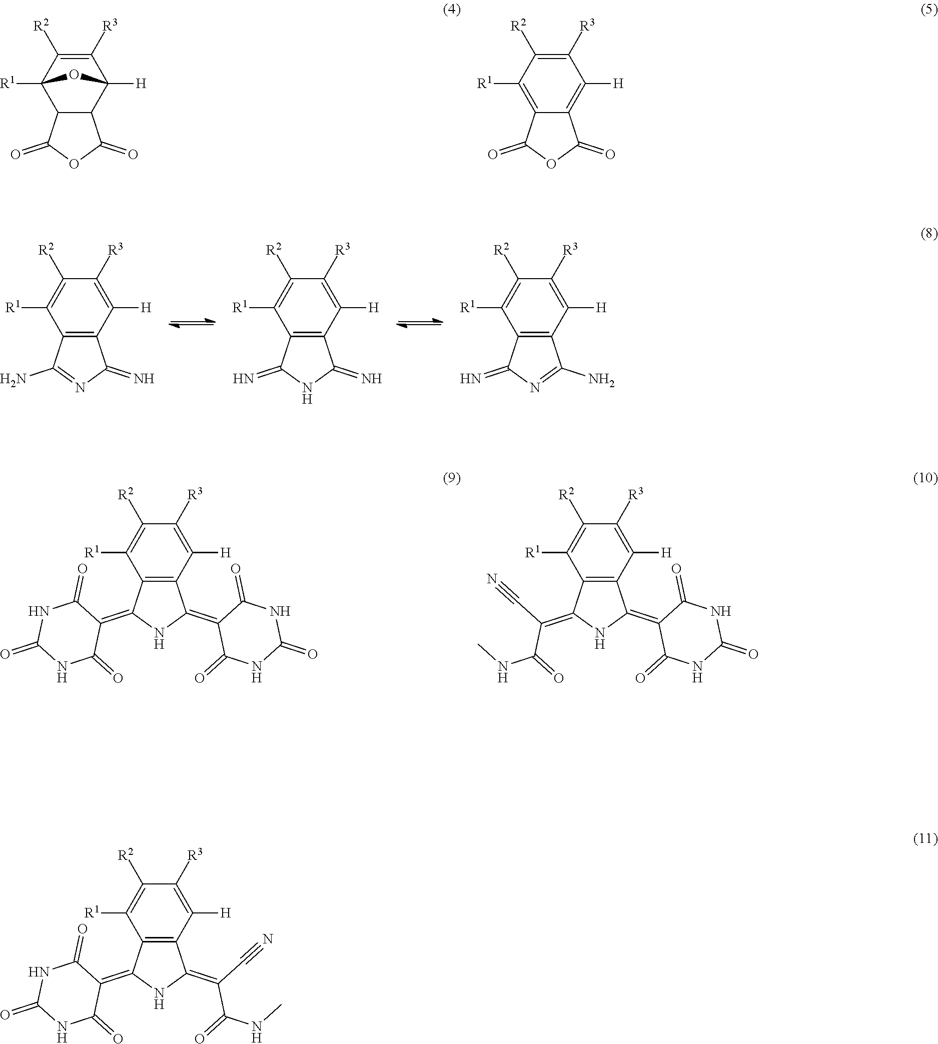

In the general formulas (4), (5), and (8)-(11), R¹ may represent a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and R² and R³ may each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or R² and R³ may be ring-closed to form a five- to eight-membered ring.

Preferred ranges and examples of R¹ to R³ may be the same as those listed above for the R¹ to R³ of the general formulas (1) and (2) in the method for producing a furoic acid derivative of an embodiment.

[Step (a)]

In the step (a), a furan derivative represented by the general formula (3) and maleic anhydride are subjected to the Diels-Alder reaction to produce a compound represented by the general formula (4) (a DA intermediate). Specific examples of the furan derivative represented by the general formula (3) are the same as those listed above.

Specific examples of the compound represented by the general formula (4) (the DA intermediate) are the same as those listed above in the step (A) of the method for producing a phthalocyanine derivative of an embodiment.

The preferred reaction solvent may be the same as those listed above for the step (A) of the method for producing a phthalocyanine derivative of an embodiment.

The preferred ranges of the reaction temperature and the reaction pressure may be the same as those listed above for the step (A) of the method for producing a phthalocyanine derivative of an embodiment.

[Step (b)]

In the step (b), by ring-opening dehydration of the compound represented by the general formula (4) (the DA intermediate) obtained in the step (a), a compound represented by the general formula (5) (a phthalic anhydride derivative) can be produced.

Specific examples of the compound represented by the general formula (5) (the phthalic anhydride derivative) are the same as those listed above in the step (B) of the method for producing a phthalocyanine derivative of an embodiment.

The preferred reaction solvent may be the same as those listed above for the step (B) of the method for producing a phthalocyanine derivative of an embodiment. The ring-opening dehydration reaction may also be carried out without a solvent.

The preferred ranges of the reaction temperature may be the same as those listed above for the step (B) of the method for producing a phthalocyanine derivative of an embodiment.

The preferred catalyst and the preferred amount thereof may be the same as those listed above for the step (B) of the method for producing a phthalocyanine derivative of an embodiment.

### [Step (C)]

In the step (C), by ring-opening dehydration of the compound represented by the general formula (5) (the phthalic anhydride derivative) obtained in the step (b) is reacted with urea or NH₃ gas and a nitrate salt of ammonium, lithium, sodium, potassium, magnesium, calcium, or aluminum in the presence of a catalyst to produce an isoindoline derivative represented by the general formula (8).

The reaction solvent is not limited as long as it allows the reaction to proceed suitably, but methanol, ethanol, isopropyl alcohol, DMSO, DMF or water is preferred.

The reaction temperature is not limited as long as it allows the reaction to proceed suitably, but the lower limit is preferably 20° C. or higher, preferably 40° C. or higher, preferably 60° C. or higher, preferably 80° C. or higher, and preferably 100° C. or higher. The upper limit is preferably 280° C. or lower, preferably 260° C. or lower, preferably 240° C. or lower, preferably 220° C. or lower, preferably 200° C. or lower, and preferably 180° C. or lower. Any single or combination of these upper and lower limits is acceptable

Urea or NH₃ gas can be used as an amine source. To replace all oxygen atoms into nitrogen atoms, and prevent side reactions such as oligomerization and polymerization, it is preferable to add a nitrate salt of ammonium, lithium, sodium, potassium, magnesium, calcium, or aluminum.

The catalyst is not limited as long as it allows the reaction to proceed suitably, but a molybdenum catalyst is preferred, and ammonium molybdate (VI) tetrahydrate is more preferred.

As for the amount of the catalyst added, the lower limit thereof is preferably 0.001 mol % or more, more preferably 0.01 mol % or more, and even more preferably 0.1 mol % or more relative to the compound represented by the general formula (5) (the phthalic anhydride derivative). The upper limit thereof is preferably 10 mol % or less, more preferably 5 mol % or less, and even more preferably 3 mol % or less. Any single or combination of these upper and lower limits is acceptable.

Further, an isoindoline derivative represented by general formulas (9) can be produced by making the obtained isoindoline derivative represented by the general formula (8) react with barbituric acid. Isoindoline derivatives represented by general formulas (10) and (11) can be produced by making the obtained isoindoline derivative represented by the general formula (8) react with barbituric acid and 2-cyano-N-methylacetamide.

The reaction between the isoindoline derivative represented by the general formula (8) and barbituric acid includes, but is not limited to, for example, the method described in The Journal of Organic Chemistry 2019, 84, 10, 6217-6222; CN103289434A; CN102585542A; JP2019112537A; or WO2009074533A2.

The reaction of the isoindoline derivative represented by the general formula (8) with barbituric acid and 2-cyano-N-methylacetamide includes, but is not limited to, for example, the method described in JP2020026503A, JP2023022808A, WO2022014635A1 or JP2022139293A.

Specific examples of the reaction solvent are the same as those listed above for the synthesis of an isoindoline derivative represented by the general formula (8).

Preferred ranges of the reaction temperature may be the same as those listed above for the synthesis of an isoindoline derivative represented by the general formula (8).

The catalyst is not limited as long as it allows the reaction to proceed suitably, but an acid such as formic acid, hydrochloric acid, nitric acid, sulfuric acid, methanesulfonic acid or p-toluenesulfonic acid is preferred, and acetic acid is more preferred in the synthesis of formula (9) to (11).

As for the amount of the catalyst added, the lower limit thereof is preferably 0.001 mol % or more, more preferably 0.01 mol % or more, and even more preferably 0.1 mol % or more relative to the compound represented by the general formula (5) (the phthalic anhydride derivative). The upper limit thereof is preferably 10 mol % or less, more preferably 5 mol % or less, and even more preferably 3 mol % or less. Any single or combination of these upper and lower limits is acceptable.

Since the raw material, a furan derivative, may be produced using a method for producing a furan derivative of an embodiment, the method for producing an isoindoline derivative of an embodiment may be simple and inexpensive and can be performed using ordinary laboratory facilities, facilitating scale-up and reducing manufacturing costs.

In a method for producing a furoic acid derivative of an embodiment, it is preferable that the raw material, a furfural derivative, is derived from biomass from the viewpoint of reducing environmental load.

In a method for producing a furan derivative of an embodiment, it is preferable that the raw material, a furfural derivative or a furoic acid derivative, is derived from biomass from the viewpoint of reducing environmental load.

In a method for producing a phthalocyanine derivative of an embodiment, it is preferable that the raw material, a furan derivative, is derived from biomass from the viewpoint of reducing environmental load.

In the present disclosure, biomass may refer to a plant as a source of alternative energy. Biomass is typically composed mainly of two components, lignin and (hemi)cellulose. Both lignin and (hemi)cellulose are polymers. Lignin may be composed of aromatic monomers, while (hemi)cellulose may be composed of 5-carbon and 6-carbon sugars. In the methods for producing furoic acid derivatives and furan derivatives of an embodiment, both lignin-derived and (hemi)cellulose-derived raw materials can be used as raw materials.

A biomass-derived furfural derivative can be produced from a (hemi)cellulose-derived sugar, for example, as described in JP5791838B.

A biomass-derived furoic acid derivative can, for example, be produced using the method for producing a furoic acid derivative of an embodiment, using a biomass-derived furfural derivative as a raw material.

A biomass-derived furan derivative can, for example, be produced using a method for producing a furan derivative of an embodiment, using a biomass-derived furfural derivative or a biomass-derived furoic acid derivative as a raw material.

The biomass degree of a furfural derivative, a furoic acid derivative, and a furan derivative, which are a raw material used for the methods for producing a furoic acid derivative, a furan derivative, and a phthalocyanine derivative of an embodiment, respectively, is preferably 1% or more, preferably 5% or more, preferably 10% or more, preferably 15% or more, preferably 20% or more, preferably 25% or more, preferably 30% or more, preferably 35% or more, preferably 40% or more, preferably 45% or more, preferably 50% or more, preferably 55% or more, preferably 60% or more, preferably 65% or more, preferably 70% or more, preferably 75% or more, preferably 80% or more, more preferably 85% or more, even more preferably 90% or more, especially preferably 95% or more. The upper limit of the biomass degree is not limited, and may be 100% or less, for example.

The biomass degree of a furoic acid derivative, a furan derivative, and a phthalocyanine derivative obtained using the methods for producing a furoic acid derivative, a furan derivative, and a phthalocyanine derivative of an embodiment, respectively, is preferably 1% or more, preferably 5% or more, preferably 10% or more, preferably 15% or more, preferably 20% or more, preferably 25% or more, preferably 30% or more, preferably 35% or more, preferably 40% or more, preferably 45% or more, preferably 50% or more, preferably 55% or more, preferably 60% or more, preferably 65% or more, preferably 70% or more, preferably 75% or more, preferably 80% or more, more preferably 85% or more, even more preferably 90% or more, especially preferably 95% or more. The upper limit of the biomass degree is not limited, and may be 100% or less, for example.

In the present disclosure, a biomass degree may be the content (% by mass) of the carbon of biomass origin (the radiocarbon atom ¹⁴C) in the total carbon, as calculated by measurement according to ASTM-D6866-18. Since the carbons in a fossil-derived compound or composition do not typically contain the radiocarbon atom ¹⁴C, the measurement of ¹⁴C can be used to confirm whether the compound or composition produced is a fossil-derived compound or a biomass-derived compound.

In the present disclosure, “containing the radiocarbon atom ¹⁴C” may include the meaning in the mass balance approach and the book-and-claim approach as well as the meaning in the segregation approach (see Enabling Circular Economy for Chemical with the Mass Balance Approach, the Ellen MacAthur Foundation network).

When a biomass-derived raw material by the mass balance method or the book-and-claim method is available, it is easier to set the biomass degree of the raw material used in the methods for producing a furoic acid derivative, a furan derivative, and a phthalocyanine derivative to be between 1% and 100%. In the present disclosure, the biomass degree including a biomass-derived raw material by the mass balance method or the book-and-claim method is covered.

### EXAMPLES

The following describes an embodiment through certain examples and comparative examples. However, specific embodiments are not limited to these examples.

The following describes measurement methods used in the examples and comparative examples.

### [Nuclear Magnetic Resonance Analysis (NMR)]

The obtained reaction product was subjected to molecular structure analysis using ¹H-NMR. By confirming the peak derived from the target product, it was confirmed that the target product was assuredly obtained. The measurement conditions were as follows:

### <Measurement Apparatus and Conditions>

Measurement apparatus: JNM-ECM400S (produced by JEOL RESONANCE Co., Ltd.).
Resonance Frequency: 400 MHz.
Accumulation count: 16 times.
Solvent: DMSO-d6.
Sample concentration: 5 mg/lmL.

### [Gas Chromatography Mass Spectrometry (GC-MS)]

Gas chromatography-mass spectrometry analysis was performed on furans synthesized in Examples 5 and 6 and on furan in Comparative Example 1. Furan was dissolved in methanol, ethanol, ether, or tetrahydrofuran to a concentration of 0.5 to 3.0 mg/mL to make the measurement solution as needed. The measurement conditions were as follows:

### <Measurement Apparatus and Conditions>

Measurement apparatus: GC7890B MSD5977B (produced by Agilent Technologies).
Column: InertCap-5MS (having an inner diameter of 0.25 mm, a length of 30 m, and a film thickness of 0.25 μm) (produced by Agilent Technologies).
Carrier gas: helium.
Carrier gas flow rate: 1.42 mL/min.
Injection volume: 1 μL.
Split ratio: 30:1.
Vaporization chamber temperature: 280° C.
Program for column temperature: the temperature was kept at 40° C. for 2 min, raised at 10° C./min, and then kept at 280° C. for 4 min.
Ion source: EI.

### Example 1

60 g of sodium phosphate and 48 g of furfural were added to a 500 mL four-necked flask equipped with a mechanical stirrer, a condenser, a dropping funnel, and a thermometer. The reaction mixture was stirred and heated to 55° C., then 170 g of 30% H₂O₂ was added dropwise over 1 hour. The reaction mixture was then held at 55° C. for 8 hours. Throughout the reaction, the pH of the reaction system was in the range of 3 to 5. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 100 mL round-bottom flask equipped with a distillation apparatus, and 0.04 g of Cu₂O and 3 mL of N-methyl-2-pyrrolidone were added. The reaction mixture was stirred and held at 185° C. for 2 hours. Finally, furan (colorless liquid) was collected (2.8 g, 8% yield).

### Example 2

101.19 g of triethylamine, 120 g of potassium acetate and 48 g were added to a 1 L four-necked flask equipped with a mechanical stirrer, a condenser, a dropping funnel, and a thermometer. The reaction mixture was stirred and heated to 55° C., then 170 g of 30% H₂O₂ was added dropwise over 1 hour. The reaction mixture was then held at 55° C. for 6 hours. Throughout the reaction, the pH of the reaction system was in the range of 9 to 12. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 300 mL round-bottom flask equipped with a distillation apparatus, and 0.32 g of Cu₂O and 25 mL of N-methyl-2-pyrrolidone were then added. The reaction mixture was stirred and held at 185° C. for 2 hours. Finally, furan (colorless liquid) was collected (22.8 g, 66% yield).

### Example 3

41 g of sodium acetate and 48 g of furfural were added to a 500 mL four-necked flask equipped with a mechanical stirrer, a condenser, a dropping funnel, and a thermometer. The reaction mixture was stirred and heated to 55° C., then 170 g of 30% H₂O₂ was added dropwise over 1 hour. The reaction mixture was then held at 55° C. for 8 hours. Throughout the reaction, the pH of the reaction system was in the range of 8 to 10. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 100 mL round-bottom flask equipped with a distillation apparatus, and 0.11 g of Cu₂O and 9 mL of N-methyl-2-pyrrolidone were added. The reaction mixture was stirred and held at 185° C. for 2 hours. Finally, furan (colorless liquid) was collected (8.2 g, 24% yield).

### Example 4

147 g of potassium acetate and 48 g of furfural were added to a 500 mL four-necked flask equipped with a mechanical stirrer, a condenser, a dropping funnel, and a thermometer. The reaction mixture was stirred and heated to 55° C., then 170 g of 30% H₂O₂ was added dropwise over 1 hour. The reaction mixture was then held at 55° C. for 8 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 100 mL round-bottom flask equipped with a distillation apparatus, and 0.28 g of Cu₂O and 22 mL of N-methyl-2-pyrrolidone were added. The reaction mixture was stirred and held at 185° C. for 2 hours. Finally, furan (colorless liquid) was collected (20 g, 58% yield).

### Example 5

74 g of potassium acetate and 48 g of furfural were added to a 500 mL four-necked flask equipped with a mechanical stirrer, a condenser, a dropping funnel, and a thermometer. The reaction mixture was stirred and heated to 75° C., then 68 g of 30% H₂O₂ was added dropwise over 1 hour. The reaction mixture was then held at 75° C. for 6 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 100 mL round-bottom flask equipped with a distillation apparatus, and 0.28 g of Cu₂O and 22 mL of N-methyl-2-pyrrolidone were added. The reaction mixture was stirred and held at 185° C. for 2 hours. Finally, furan (colorless liquid) was collected (20.6 g, 60% yield).

The purity of furoic acid determined from the GC-MS readings was >99.5%, and <0.5% of furanone, fumaric acid, maleic acid or succinic acid was contained. The purity of furan determined from the GC-MS readings was 99.844%, and 0.156% of 2-methylfuran was contained.

### Example 6

74 g of potassium acetate and 48 g of furfural were added to a 500 mL four-necked flask equipped with a mechanical stirrer, a condenser, a dropping funnel, and a thermometer. The reaction mixture was stirred and heated to 75° C., then 68 g of 30% H₂O₂ was added dropwise over 1 hour. The reaction mixture was then held at 75° C. for 6 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 200 mL round-bottom flask equipped with a distillation apparatus, and 0.29 g of Cu₂O and 45 mL of N-ethyl-2-pyrrolidone were added. The reaction mixture was stirred and held at 185° C. for 2 hours. Finally, furan (colorless liquid) was collected (20.6 g, 60% yield).

The purity of furoic acid determined from the GC-MS readings was >99.5%, and <0.5% of furanone, fumaric acid, maleic acid or succinic acid was contained. The purity of furan determined from the GC-MS readings was 99.880%, and 0.120% of 2-methylfuran was contained.

### Example 7

74 g of potassium acetate and 48 g of furfural were added to a 500 mL four-necked flask equipped with a mechanical stirrer, a condenser, a dropping funnel, and a thermometer. The reaction mixture was stirred and heated to 75° C., then 68 g of 30% H₂O₂ was added dropwise over 1 hour. The reaction mixture was then held at 75° C. for 6 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 100 mL round-bottom flask equipped with a distillation apparatus, and was stirred and held at 185° C. for 2 hours. Finally, furan (colorless liquid) was collected (18.1 g, 52% yield).

### Example 8

74 g of potassium acetate and 48 g of furfural were added to a 500 mL four-necked flask equipped with a mechanical stirrer, a condenser, a dropping funnel, and a thermometer. The reaction mixture was stirred and heated to 75° C., then 68 g of 30% H₂O₂ was added dropwise over 1 hour. The reaction mixture was then held at 75° C. for 6 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 200 mL round-bottom flask equipped with a distillation apparatus, and 45 mL of N-ethyl-2-pyrrolidone was added. The reaction mixture was stirred and held at 185° C. for 2 hours. Finally, furan (colorless liquid) was collected (11.1 g, 32% yield).

### Example 9

74 g of potassium acetate and 48 g of furfural were added to a 500 mL four-necked flask equipped with a mechanical stirrer, a condenser, a dropping funnel, and a thermometer. The reaction mixture was stirred and heated to 75° C., then 68 g of 30% H₂O₂ was added dropwise over 1 hour. The reaction mixture was then held at 75° C. for 6 hours. Throughout the reaction, the pH of the reaction system was in the range of 9 to 11. The reaction mixture was then cooled to room temperature, brought to a pH of 12 using NaOH solution, and extracted with ethyl acetate. The resulting extract was acidified with hydrochloric acid and filtered to collect furoic acid (white solid).

The collected furoic acid was placed in a 200 mL round-bottom flask equipped with a distillation apparatus, and 57 mL of hexadecane was added. The reaction mixture was stirred and held at 185° C. for 2 hours. Finally, furan (colorless liquid) was collected (9.7 g, 35% yield).

### Comparative Example 1

Commercially available furan (produced by Sinopharm Chemical Reagent Co., Ltd.) was used as Comparative Example 1 and compared to the examples.

The purity of furan determined from GC-MS readings was 100.000%.

### INDUSTRIAL APPLICABILITY

According to the present disclosure, furoic acid derivatives, furan derivatives, phthalocyanine derivatives, and isoindoline derivatives can be produced in higher yields and at lower cost, respectively, using a simpler production method than in the past. Therefore, each of the production methods of the present disclosure can be easily scaled up and is a clean and green method that can greatly contribute to the future development of biomass-derived production of furoic acid derivatives, furan derivatives, phthalocyanine derivatives, and isoindoline derivatives.

1. A method for producing a furoic acid derivative, comprising an oxidation step of oxidizing a furfural derivative represented by general formula (1) to obtain a furoic acid derivative represented by general formula (2) as shown in reaction formula (I), wherein a pH of the reaction system is from 3 to 12, 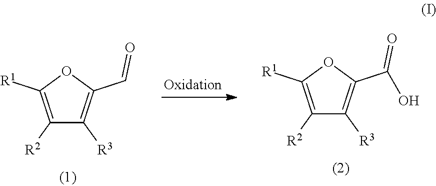
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

2. The method for producing a furoic acid derivative according to claim 1, wherein one or more compounds selected from a group consisting of formic acid, hydrochloric acid, nitric acid, sulfuric acid, methanesulfonic acid, p-toluenesulfonic acid, phosphoric acid, phosphoric anhydride, polyphosphoric acid, pyrophosphoric acid, phosphorous acid, and salts thereof, and compounds represented by general formulas (A1) to (A4) are used as an additive in the oxidation step, 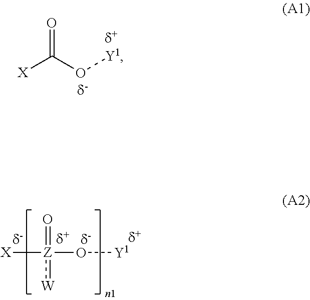
wherein,
X represents an —OH group, an —OM¹ group or an —R⁴,
wherein M¹ represents an alkali metal atom, and
R⁴ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Y¹ represents a hydrogen atom, an alkali metal atom, —COR⁵ or a functional group represented by general formula (B),
wherein R⁵ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Z represents a sulfur atom or a phosphorus atom,
W represents an oxygen atom or is roughly equivalent to the X,
n1 is an integer from 1 to 10, 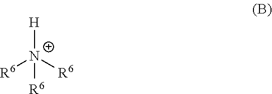
R⁶ in general formula (B) represents a hydrogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, 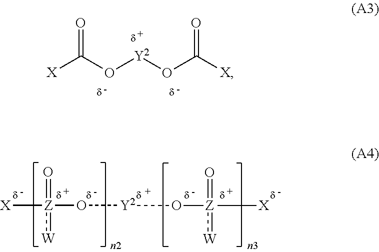
X in general formulas (A3) and (A4) each independently are roughly equivalent to the X in the general formulas (A1) and (A2),
Y² represents an alkaline earth metal atom,
Z is roughly equivalent to the Z in the general formulas (A1) and (A2)
W represents an oxygen atom or is roughly equivalent to the X in the general formulas (A1) and (A2), and
n2 and n3 each, independently, is an integer from 1 to 10.

3. The method for producing a furoic acid derivative according to claim 1, wherein O₂, H₂O₂, O₃, KMnO₄, KClO₃, or NaClO is used as an oxidant in the oxidation step.

4. The method for producing a furoic acid derivative according to claim 1, wherein the oxidation step is performed at a temperature of 0 to 120° C.

5. A method for producing a furan derivative, comprising a decarboxylation step of decarboxylating a furoic acid derivative represented by general formula (2) to obtain a furan derivative represented by general formula (3) as shown in reaction formula (II), wherein decarboxylation is performed without a catalyst or using an oxide of mono- to tri-valent transition metal as a catalyst, 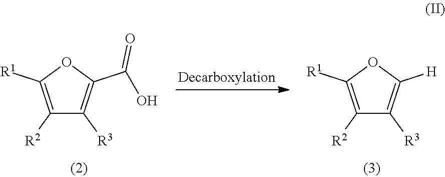
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

6. A method for producing a furan derivative, comprising a decarboxylation step of decarboxylating a furoic acid derivative represented by general formula (2) to obtain a furan derivative represented by general formula (3) as shown in reaction formula (II), wherein decarboxylation is performed using a high boiling point solvent, 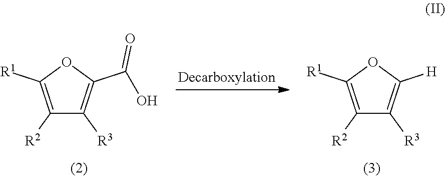
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

7. A method for producing a furan derivative, comprising
an oxidation step of oxidizing a furfural derivative represented by general formula (1) to obtain a furoic acid derivative represented by general formula (2) as shown in reaction formula (III), wherein a pH of the reaction system is from 3 to 12; and
a decarboxylation step of decarboxylating the furoic acid derivative represented by the general formula (2) obtained in the oxidation step to obtain a furan derivative represented by general formula (3) as shown in reaction formula (III), wherein decarboxylation is performed without a catalyst or using an oxide of a mono- to tri-valent transition metal as a catalyst, 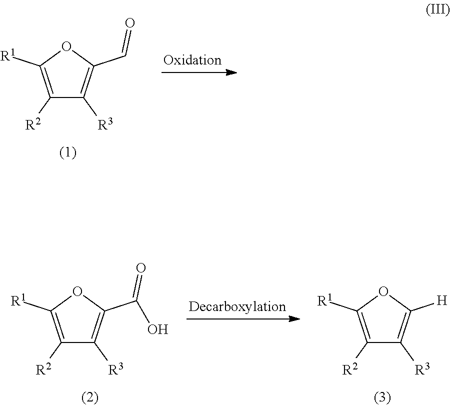
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

8. A method for producing a furan derivative, comprising
an oxidation step of oxidizing a furfural derivative represented by general formula (1) to obtain a furoic acid derivative represented by general formula (2) as shown in reaction formula (III), wherein a pH of the reaction system is from 3 to 12; and
a decarboxylation step of decarboxylating the furoic acid derivative represented by the general formula (2) obtained in the oxidation step to obtain a furan derivative represented by general formula (3) as shown in reaction formula (III), wherein decarboxylation is performed using a high boiling point solvent. 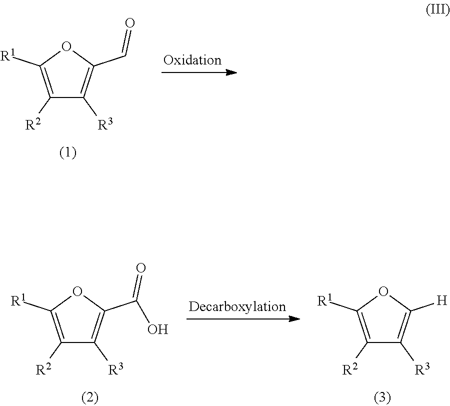
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.

9. The method for producing a furan derivative according to claim 7, wherein one or more compounds selected from a group consisting of formic acid, hydrochloric acid, nitric acid, phosphoric acid, phosphoric anhydride, polyphosphoric acid, pyrophosphoric acid, sulfuric acid, methanesulfonic acid, p-toluenesulfonic acid, phosphorous acid, and salts thereof, and compounds represented by general formulas (A1) to (A4) are used as an additive(s) in the oxidation step, 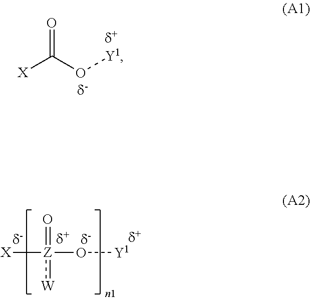
wherein,
X represents an —OH group, an —OM¹ group or an —R⁴,
wherein M¹ represents an alkali metal atom, and
R⁴ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Y¹ represents a hydrogen atom, an alkali metal atom, —COR⁵ or a functional group represented by general formula (B),
wherein R⁵ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
Z represents a sulfur atom or a phosphorus atom,
W represents an oxygen atom or is roughly equivalent to the X,
n1 is an integer from 1 to 10, 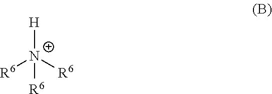
R⁶ in general formula (B) represents a hydrogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, 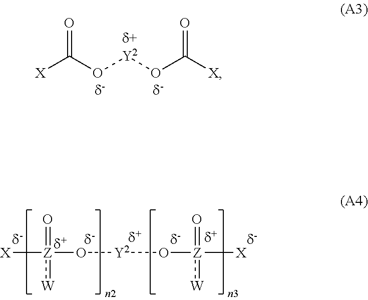
X in general formulas (A3) and (A4) each independently are roughly equivalent to the X in the general formulas (A1) and (A2),
Y² represents an alkaline earth metal atom,
Z is roughly equivalent to the Z in the general formulas (A1) and (A2)
W represents an oxygen atom or is roughly equivalent to the X in the general formulas (A1) and (A2), and
n2 and n3 each, independently, is an integer from 1 to 10.

10. The method for producing a furan derivative according to claim 7, wherein O₂, H₂O₂, O₃, KMnO₄, KClO₃, or NaClO is used as an oxidant in the oxidation step.

11. The method for producing a furan derivative according to claim 7, wherein the oxidation step is performed at a temperature from 0 to 120° C.

12. The method for producing a furan derivative according to claim 5, wherein a biomass degree of the furan derivative is 1% or more.

13. A method for producing a phthalocyanine derivative from the furan derivative obtained by the method for producing a furan derivative according to claim 5, comprising
a step (A) of obtaining a compound represented by general formula (4) from the furan derivative represented by the general formula (3);
a step (B) of obtaining a compound represented by general formula (5) from the compound represented by the general formula (4) obtained in the step (A); and
a step (C) of obtaining a phthalocyanine derivative represented by general formula (6) or (7) from the compound represented by the general formula (5) obtained in the step (B) 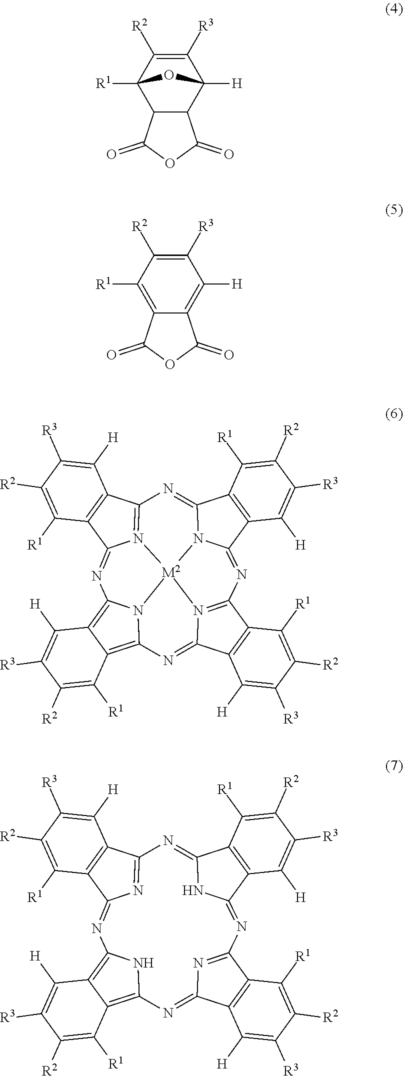
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms,
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
And M² represents a metal atom.

14. A method for producing an isoindoline derivative from the furan derivative obtained by the method for producing a furan derivative according to claim 5, comprising
a step (a) of obtaining a compound represented by general formula (4) from the furan derivative represented by the general formula (3);
a step (b) of obtaining a compound represented by general formula (5) from the compound represented by the general formula (4) obtained in the step (a); and
a step (C) of obtaining at least one of isoindoline derivatives represented by general formulas (8) to (11) from the compound represented by the general formula (5) obtained in the step (b) 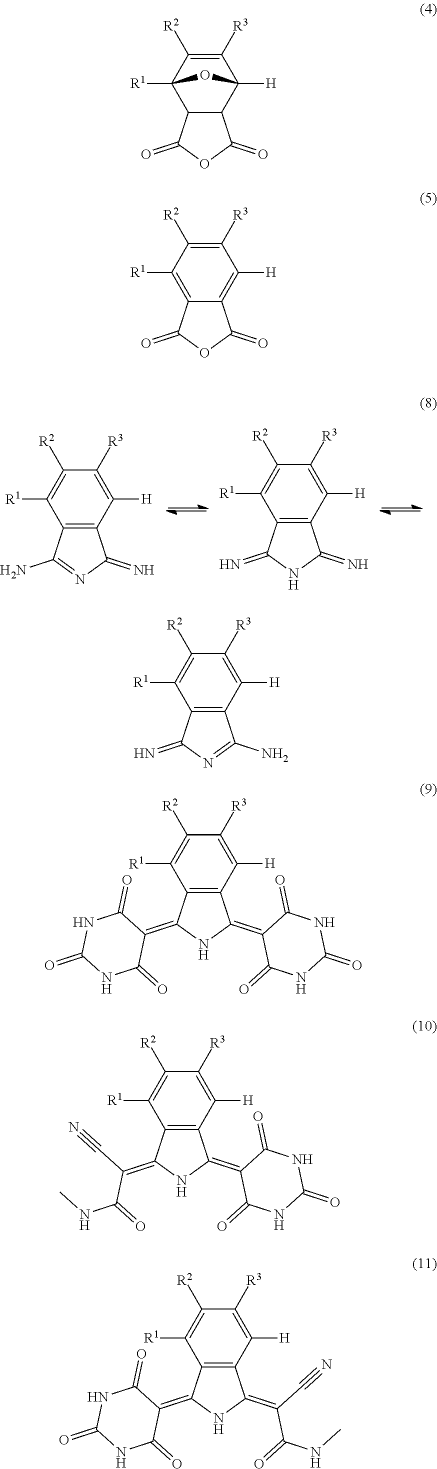
and wherein,
R¹ represents a hydrogen atom, a halogen atom, an optionally substituted linear or branched alkyl group having 1 to 28 carbon atoms, an optionally substituted cycloalkyl group having 3 to 7 carbon atoms, or an optionally substituted aryl group having 6 to 12 carbon atoms, and
R² and R³ each, independently represents a hydrogen atom, a halogen atom, an optionally substituted linear or branched alkyl group having 1 to 28 carbon atoms, an optionally substituted cycloalkyl group having 3 to 7 carbon atoms, an optionally substituted aryl group having 6 to 12 carbon atoms, or
R² and R³ are ring-closed to form a five- to eight-membered ring.
